(19) United States
(12) Patent Application Publication
(10) Pub. No.: US 2026/0131121 A1
(43) Pub. Date: May 14, 2026

(54) MICRONEEDLE PATCH

(71) Applicant: DARWIN PRECISIONS CORPORATION, Hsinchu County (TW)

(72) Inventors: Chung-Lin Lee, Hsinchu County (TW); Yun-Pei Yang, Hsinchu County (TW)

(21) Appl. No.: 19/256,209

(22) Filed: Jul. 1, 2025

(30) Foreign Application Priority Data
Nov. 11, 2024 (TW) 113143214

(51) Int. Cl. A61M 37/00

(52) U.S. Cl. CPC A61M 37/0015; A61M 2037/0023; A61M 2205/02

(57) ABSTRACT

A microneedle patch includes a base layer and microneedle structures. The base layer has a first surface, and the base layer is made of a base material. The microneedle structures are disposed on the first surface, and each microneedle structure includes a microneedle outer layer and a microneedle inner layer. The microneedle outer layer connected to the first surface is made of an outer-layer material, and has a microneedle cavity; the microneedle inner layer is made of an inner-layer material, and is arranged in the microneedle cavity. The base material and the outer-layer material include at least one hydrophilic material, and the inner-layer material includes at least one solid grease. The microneedle patch uses solid oil to carry fat-soluble functional ingredients and allows the fat-soluble functional ingredients to be smoothly transported into the user's body.

### FIELD OF THE INVENTION

The present invention relates to a microneedle patch, and more particularly to a microneedle patch with fat-soluble functional ingredients.

### BACKGROUND OF THE INVENTION

Skin films in the cosmetics market are mainly divided into dry and wet types. Among them, the dry-type microneedle patches (MNPs) are a new type of transdermal drug delivery system (TDDS) that combines the advantages of patches and subcutaneous injections. Since the microneedles on the patches are very short, they don't touch the nerves and cause pain like subcutaneous injections. In addition, they can easily transport the macromolecular medicines through the skin's stratum corneum into the human body. In comparison with the wet-type films, the dry-type microneedle patches can deliver active ingredients of medicines faster and more effectively. However, the base materials of the dry-type microneedle patches are mainly made of water-soluble ingredients, such as hyaluronic acid, polyvinyl pyrrolidone, polyvinyl alcohol or carboxymethyl cellulose. Thus, when some medicines or ingredients are fat-soluble, the microneedles are prone to meet some problems, such as oil-water separation, non-uniform distribution of the fat-soluble ingredients in the microneedles, failure to form microneedles, or fragile microneedles, during the microneedles manufacturing process. Therefore, developing a microneedle patch capable of delivering fat-soluble medicines is necessary.

### SUMMARY OF THE INVENTION

The present invention provides a microneedle patch having a microneedle outer layer and a microneedle inner layer with materials of different properties so as to carry fat-soluble ingredients and achieve the goal of delivering the fat-soluble ingredients into the human body.

In order to achieve one, part or all of the above objects or other objects, the present invention provides a microneedle patch including a base layer and a plurality of microneedle structures. The base layer has a first surface, and the base layer is made of a base material. Each of the microneedle structures is disposed on the first surface and includes a microneedle outer layer and a microneedle inner layer. The microneedle outer layer connected to the first surface is made of an outer-layer material, and has a microneedle cavity. The microneedle inner layer is made of an inner-layer material, and is arranged in the microneedle cavity. The base material and the outer-layer material include at least one hydrophilic material, and the inner-layer material includes at least one solid grease.

In one embodiment of the present invention, the hydrophilic material is selected from the group consisting of hyaluronic acid, polyvinyl pyrrolidone, polyvinyl alcohol, hydroxyethyl cellulose, carboxymethyl cellulose, hydroxypropyl methylcellulose, pullulan gum, dextran, collagen, xanthan gum, pectin, polyglutamic acid, sodium alginate, maltodextrin, cyclodextrin, propylene glycol alginate, polymannose, trehalose, gelatin, gum arabic, and methylcellulose.

In one embodiment of the present invention, the solid grease is selected from the group consisting of cocoa butter, hydrogenated cocoa butter, triglycerides, palm fat, beeswax, microcrystalline wax, shea butter, carnauba wax, cetyl alcohol, stearyl alcohol, stearic acid, lauric acid, palmitic acid, cinnamic acid, coconut oil, lard, tallow, lanolin, horse oil, sea otter oil, whale oil, and cetearyl alcohol.

In one embodiment of the present invention, the inner-layer material further includes a fat-soluble functional ingredient selected from the group consisting of vitamin C palmitate, mandelic acid, sulfur, salicylic acid, tocopherol, plant essential oils, plant functional oils, coenzyme Q10, retinol, ceramide, lutein, totarol, thyroxine, vitamin A, vitamin D, vitamin K, and any combination thereof.

In one embodiment of the present invention, the fat-soluble functional ingredient and the at least one solid grease of the inner-layer material are mixed uniformly. A mass ratio of the fat-soluble functional ingredient to the at least one solid grease of the inner-layer material is more than or equal to 0.05:1 and less than 1:1.

In one embodiment of the present invention, a melting point of the inner-layer material is 25° C. to 40° C.

In one embodiment of the present invention, the microneedle structure has a microneedle height between 100 μm and 1500 μm, and the base layer has a base-layer height between 5 μm and 300 μm.

In one embodiment of the present invention, the microneedle outer layer has an outer surface, the microneedle cavity has a sidewall, and the microneedle inner layer has a side surface. The side surface of the microneedle inner layer is adjoined to the sidewall of the microneedle cavity, and a wall thickness between the outer surface and the sidewall is between 5 μm and 20 μm.

In one embodiment of the present invention, a cross-sectional area of the microneedle outer layer parallel to the first surface has a largest outer-layer width from 260 μm to 540 μm, and a cross-sectional area of the microneedle inner layer parallel to the first surface has a largest inner-layer width from 250 μm to 500 μm.

In one embodiment of the present invention, the microneedle structures are pyramids or cones.

The present invention provides a microneedle outer layer made of a hydrophilic material and a microneedle inner layer made of solid grease. Thus, the microneedle patch is cable of transporting fat-soluble ingredients, and the microneedle structures are stable and not easy to disintegrate.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic cross-sectional view of a microneedle patch according to an embodiment of the invention.

FIG. 2 is a perspective schematic view of a microneedle patch according to an embodiment of the invention.

FIG. 3 is a perspective schematic view of a microneedle patch according to another embodiment of the invention.

### DETAILED DESCRIPTION OF PREFERRED EMBODIMENTS

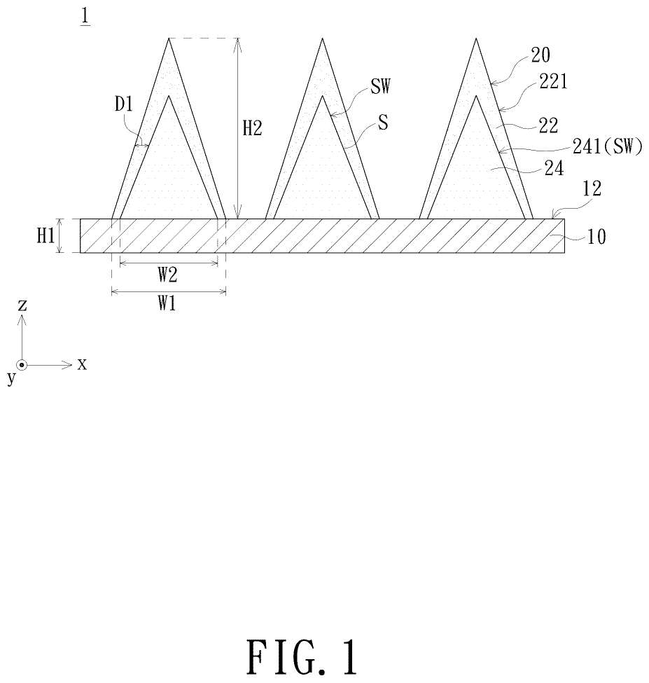

FIG. 1 is a schematic cross-sectional view of a microneedle patch according to an embodiment of the invention. As shown in FIG. 1, a microneedle patch 1 includes a base layer 10 and a plurality of microneedle structures 20. The base layer 10 has a first surface 12, and the base layer 10 is made of a base material. Each of the microneedle structures 20 is disposed on the first surface 12 and includes a microneedle outer layer 22 and a microneedle inner layer 24. The microneedle outer layer 22 is made of an outer-layer material and connected to the first surface 12. The microneedle outer layer includes a microneedle cavity S. The microneedle inner layer 24 is made of an inner-layer material and arranged in the microneedle cavity S. The base material and the outer-layer material include at least one hydrophilic material. The inner-layer material includes at least one solid grease. In one embodiment, the microneedle outer layer 22 has an outer surface 221, the microneedle cavity S has a sidewall SW, and the microneedle inner layer 24 has a side surface 241. The side surface 241 of the microneedle inner layer 24 is adjoined to the sidewall SW of the microneedle cavity S.

The base layer 10 of the microneedle patch 1 has a base height H1 from 5 μm to 300 μm. Each of the microneedle structures 20 has a microneedle height H2. In order to deliver the ingredients contained in the microneedle structures 20 through the skin into a user's body without stimulating the pain receptors in the dermis, the microneedle height H2 is between 100 μm and 1500 μm.

In the microneedle structure 20, the microneedle outer layer has a wall thickness D1. In an embodiment, the wall thickness D1 is defined as an interval between the outer surface 221 of the microneedle outer layer 22 and the sidewall SW of the microneedle cavity S. For example, the wall thickness D1 is defined as an interval along the x-axis between the outer surface 221 of the microneedle outer layer 22 and the sidewall SW of the microneedle cavity S, but is not limited thereto. Since the microneedle cavity S is within the microneedle outer layer 22, the microneedle cavity S would become too small if the wall thickness D1 is in too large, leading to a challenge of forming the microneedle inner layer 24. On the contrary, if the wall thickness D1 is too small, when the microneedle inner layer 24 in the microneedle cavity S melts due to a high temperature, the microneedle structure 20 tends to collapse or disintegrate, causing the microneedle inner layer 24 to overflow. Thus, the wall thickness D1 of the microneedle outer layer 22 is between 5 μm and 20 μm. In an embodiment, the wall thickness D1 along the z-axis is uniform. In another embodiment, the wall thickness D1 varies along the z-axis. For example, the wall thickness D1 may keep increasing when it leaves the first surface 12 along the z-axis or keep decreasing when it leaves the first surface 12 along the z-axis.

In one embodiment, a cross-sectional area of the microneedle outer layer 22 parallel to the first surface 12 has a largest outer-layer width W1 from 260 μm to 540 μm, and a cross-sectional area of the microneedle inner layer 24 parallel to the first surface 12 has a largest inner-layer width from 250 μm to 500 μm.

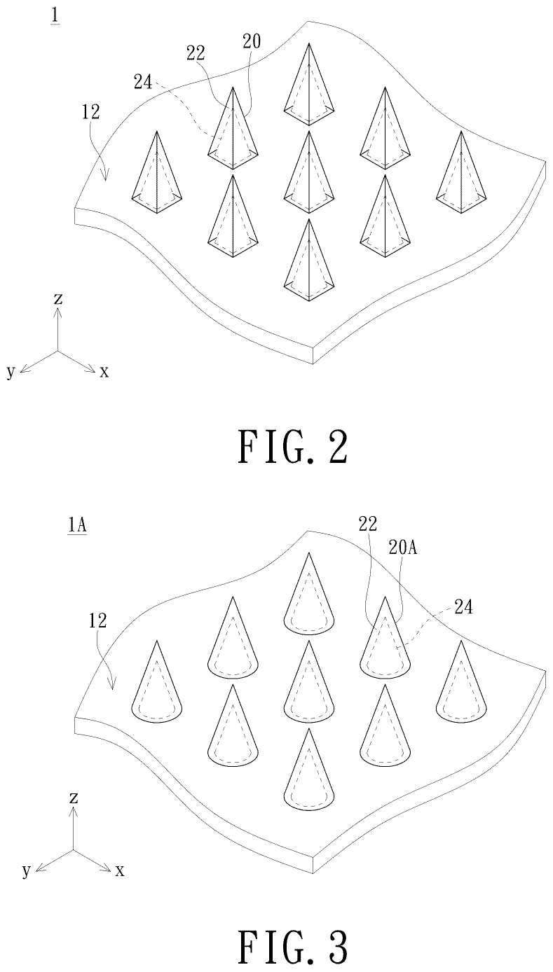

FIG. 2 is a perspective schematic view of a microneedle patch according to an embodiment of the invention. FIG. 3 is a perspective schematic view of a microneedle patch according to another embodiment of the invention. Here, the only difference between the microneedle patch 1 in FIG. 2 and the microneedle patch 1A in FIG. 3 is the different shapes of microneedle structures 20 and 20A. As shown in FIG. 2 and FIG. 3, the microneedle structures 20/20A are distributed uniformly on the first surface 12 of the base layer 10 and are arranged in an array on the first surface 12. However, the present invention is not limited thereto. In one embodiment, the microneedle structures 20/20A can be arranged alternately on the first surface 12. In addition, the microneedle structure 20 in FIG. 2 has a shape of a square pyramid, but is not limited thereto. The microneedle structure 20 may have other shapes, such as any kinds of pyramids. In one embodiment, the microneedle structure 20A has a conical shape, as illustrated in FIG. 3.

In one embodiment, the hydrophilic material contained in the base material and the outer-layer material may be selected from the group consisting of hyaluronic acid, polyvinyl pyrrolidone, polyvinyl alcohol, hydroxyethyl cellulose, carboxymethyl cellulose, hydroxypropyl methylcellulose, pullulan gum, dextran, collagen, xanthan gum, pectin, polyglutamic acid, sodium alginate, maltodextrin, cyclodextrin, propylene glycol alginate, polymannose, trehalose, gelatin, gum arabic, and methylcellulose. The base material and the outer-layer material can be the same or different.

In one embodiment, the solid grease contained in the inner-layer material may be selected from the group consisting of cocoa butter, hydrogenated cocoa butter, triglycerides, palm fat, beeswax, microcrystalline wax, shea butter, carnauba wax, cetyl alcohol, stearyl alcohol, stearic acid, lauric acid, palmitic acid, cinnamic acid, coconut oil, lard, tallow, lanolin, horse oil, sea otter oil, whale oil, and cetearyl alcohol.

In one embodiment, the inner-layer material further includes a fat-soluble functional ingredient, such as a fat-soluble medicine or vitamin, but is not limited thereto. The fat-soluble functional ingredient is selected from the group consisting of vitamin C palmitate, mandelic acid, sulfur, salicylic acid, tocopherol, plant essential oils, plant functional oils, coenzyme Q10, retinol, ceramide, lutein, totarol, thyroxine, vitamin A, vitamin D, vitamin K, and any combination thereof.

In one embodiment, when the inner-layer material is formed by a fat-soluble functional ingredient and at least one solid grease mixed uniformly, the mass ratio of the fat-soluble functional ingredient to the solid grease is determined based on the fat-soluble functional ingredient's solubility in the solid grease. For example, the mass ratio of the fat-soluble functional ingredient to the solid grease is more than or equal to 0.05:1 or less than 1:1, but is not limited thereto. In order to cure the microneedle patch 1/1A under a room temperature and ensure that the microneedle inner layer 24 can be melt properly to facilitate the delivery when the microneedle patch 1/1A is applied to a user's skin, the inner-layer material, which is mixed uniformly, has a melting point in a range from 25° C. to 40° C., preferably from 30° C. to 40° C.

Table 1 shows the results according to various mass ratios of the fat-soluble functional ingredient to the solid grease in the inner-layer material according to an embodiment of the invention. Here, the total mass of the fat-soluble functional ingredient and the solid grease is less than or equal to the total mass of the inner-layer material.

TABLE 1

| Fat-soluble | | | |
|---|---|---|---|
| functional | | | |
| ingredient A | Solid grease B | Mass Ratio | |
| Mass (%) | Mass (%) | (A:B) | Result |
| plant essential | cocoa butter 10% | 9.00:1.00 | incurable |
| oils 90% | | | |
| plant essential | cocoa butter 20% | 4.00:1.00 | incurable |
| oils 80% | | | |
| plant essential | cocoa butter 30% | 2.34:1.00 | incurable |
| oils 70% | | | |
| plant essential | cocoa butter 40% | 1.50:1.00 | incurable |
| oils 60% | | | |
| plant essential | cocoa butter 50% | 1.00:1.00 | curable |
| oils 50% | | | |
| plant essential | cocoa butter 60% | 0.67:1.00 | curable |
| oils 40% | | | |
| plant essential | cocoa butter 70% | 0.43:1.00 | curable |
| oils 30% | | | |
| plant essential | cocoa butter 80% | 0.25:1.00 | curable |
| oils 20% | | | |
| plant essential | cocoa butter 90% | 0.11:1.00 | curable |
| oils 10% | | | |
| plant essential | cocoa butter 95% | 0.05:1.00 | curable |
| oils 5% | | | |

As shown in Table 1, in an exemplary embodiment using the plant essential oils as the fat-soluble functional ingredient and using the cocoa butter as the solid grease, a mass percentage of the plant essential oils decreases from 90% to 5% in turn, and a mass percentage of the cocoa butter increases from 10% to 95% in turn correspondingly. Here, the mass ratio of the plant essential oils to the cocoa butter is between 0.05:1.00 and 9.00:1.00. In other words, the mass of the plant essential oils is between 0.05 and 9 times of that of cocoa butter.

According to the results shown in Table 1, when the mass percentage of the fat-soluble functional ingredient is too high, specifically when the mass ratio of the plant essential oils to the cocoa butter is larger than 1:1, the melting point of the microneedle inner layer 24 of the microneedle structure 20 falls below 25° C. As a result, the microneedle inner layer 24 becomes incurable, leading to an unsuccessful formation of the microneedle structures 20. When the mass ratio of the plant essential oils to the cocoa butter is between 0.05:1.00 and 1.00:1.00, the microneedle inner layer 24 can be cured successfully in the microneedle cavity S to form conical or pyramidal structures. In addition, the microneedle inner layer 24 is able to melt successfully under an environmental temperature between 25° C. and 40° C.

Table 2 shows the results according to various mass ratios of the fat-soluble functional ingredient to the solid grease in the inner-layer material according to another embodiment of the invention. Here, a total mass of the fat-soluble functional ingredient and the solid grease is less than or equal to the total mass of the inner-layer material.

TABLE 2

| | | | First mixed grease | |
|---|---|---|---|---|
| Fat-soluble | | | (hydrogenated | |
| functional | Solid | | cocoa butter to | |
| ingredient A | grease B | Mass ratio | microcrystalline | |
| Mass (%) | Mass (%) | (A:B) | wax) | Result |
| plant | First mixed | 0.67:1.00 | 1.0:1.0 | curable |
| essential | grease 60% | | | |
| oils 40% | | | | |
| plant | First mixed | 0.43:1.00 | 2.33:1.0 | curable |
| essential | grease 70% | | | |
| oils 30% | | | | |
| plant | First mixed | 0.25:1.00 | 4.0:1.0 | curable |
| essential | grease 80% | | | |
| oils 20% | | | | |
| plant | First mixed | 0.11:1.00 | 9.0:1.0 | curable |
| essential | grease 90% | | | |
| oils 10% | | | | |
| plant | First mixed | 0.05:1.00 | 9.5:1.0 | curable |
| essential | grease 95% | | | |
| oils 5% | | | | |

As shown in Table 2, in order to successfully cure the microneedle inner layer 24 in the microneedle cavity S, form conical or pyramidal structures, and melt under an environmental temperature between 25° C. and 40° C. successfully, the inner-layer material can comprise more than one solid grease, such as the first mixed grease consisting of the hydrogenated cocoa butter and the microcrystalline wax. In this embodiment, the mass ratio of the hydrogenated cocoa butter to the microcrystalline wax is between 1.0:1.0 and 9.5:1.0, and the melting point is between 25° C. and 40° C. Here, the melting points of the plant essential oils and the hydrogenated cocoa butter are both lower than that of the microcrystalline wax. Thus, both of them can help to lower the melting point of the inner-layer material in this formula.

Table 3 shows the results according to various mass ratios of the fat-soluble functional ingredient to the solid grease in the inner-layer material according to yet another embodiment of the invention. Here, the total mass of the fat-soluble functional ingredient and the solid grease is less than or equal to the total mass of the inner-layer material.

TABLE 3

| Fat-soluble | | | Second mixed | |
|---|---|---|---|---|
| functional | Solid | | grease | |
| ingredient A | grease B | Mass ratio | (triglycerides to | |
| Mass (%) | Mass (%) | (A:B) | shea butter oil) | Result |
| plant | Second mixed | 0.67:1.00 | 0.05:1.00 | curable |
| essential | grease 60% | | | |
| oils 40% | | | | |
| plant | Second mixed | 0.43:1.00 | 0.23:1.00 | curable |
| essential | grease 70% | | | |
| oils 30% | | | | |
| plant | Second mixed | 0.25:1.00 | 0.40:1.00 | curable |
| essential | grease 80% | | | |
| oils 20% | | | | |
| plant | Second mixed | 0.11:1.00 | 0.43:1.00 | curable |
| essential | grease 90% | | | |
| oils 10% | | | | |
| plant | Second mixed | 0.05:1.00 | 0.67:1.00 | curable |
| essential | grease 95% | | | |
| oils 5% | | | | |

As shown in Table 3, the mass ratio of the fat-soluble functional ingredient to the solid grease is between 0.05:1.00 and 1.00:1.00. The melting point of the inner-layer material is between 25° C. and 40° C. In the embodiment, the inner-layer material comprises a second mixed grease consisting of the triglycerides and the shea butter oil, and the mass ratio of the triglycerides to the shea butter oil is between 0.05:1.0 and 0.67:1.0.

According to the results shown in Table 1, Table 2, and Table 3, considering the content of the fat-soluble functional ingredient in the microneedle patch 1/1A and the solid grease applicable to the fat-soluble functional ingredient, a mass ratio of the fat-soluble functional ingredient to the solid grease between 0.05:1.00 and 1.00:1.00 can be obtained. In addition, in order to ensure that the melting point of the uniformly mixed inner-layer material falls between 25° C. and 40° C., a solid grease with a proper melting point range can be selected. It can also be achieved by mixing different solid greases with varying melting points based on the melting point of the fat-soluble functional ingredient, such as, but not limited to, the first mixed grease or the second mixed grease so as to keep the melting point of the mixed inner-layer material between 25° C. and 40° C. In an embodiment, the fat-soluble functional ingredient is co-enzyme Q10, and the solid grease can be selected from animal fats, nut oils, coconut oil or a combination thereof, but is not limited thereto.

Since the present invention utilizes a hydrophilic material as the microneedle outer layer and solid grease as the microneedle inner layer, the microneedle patch can effectively carry fat-soluble functional ingredients. By controlling the melting point of the inner-layer material, the fat-soluble functional ingredients, such as drugs or vitamins, can be delivered through the skin into the user's body. Furthermore, when the solid grease melts, the hydrophilic microneedle outer layer remains a stable structure, preventing the microneedle structure from collapsing and avoiding leakage of the fat-soluble functional ingredients from the microneedle inner layer. In comparison with the conventional microneedle patches, the microneedle patch of the present invention can offer a broader scope of applications.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A microneedle patch, comprising:
a base layer, having a first surface, and the base layer being made of a base material; and
a plurality of microneedle structures, disposed on the first surface, and each of the microneedle structures comprising:
a microneedle outer layer, made of an outer-layer material and connected to the first surface, and the microneedle outer layer comprising a microneedle cavity; and
a microneedle inner layer, made of an inner-layer material and disposed within the microneedle cavity;
wherein the base material and the outer-layer material comprise at least one hydrophilic material, and the inner-layer material comprises at least one solid grease.

2. The microneedle patch according to claim 1, wherein the at least one hydrophilic material is selected from the group consisting of hyaluronic acid, polyvinyl pyrrolidone, polyvinyl alcohol, hydroxyethyl cellulose, carboxymethyl cellulose, hydroxypropyl methylcellulose, pullulan gum, dextran, collagen, xanthan gum, pectin, polyglutamic acid, sodium alginate, maltodextrin, cyclodextrin, propylene glycol alginate, polymannose, trehalose, gelatin, gum arabic, and methylcellulose.

3. The microneedle patch according to claim 1, wherein the at least one solid grease is selected from the group consisting of cocoa butter, hydrogenated cocoa butter, triglycerides, palm fat, beeswax, microcrystalline wax, shea butter, carnauba wax, cetyl alcohol, stearyl alcohol, stearic acid, lauric acid, palmitic acid, cinnamic acid, coconut oil, lard, tallow, lanolin, horse oil, sea otter oil, whale oil, and cetearyl alcohol.

4. The microneedle patch according to claim 3, wherein the inner-layer material further comprises a fat-soluble functional ingredient selected from the group consisting of vitamin C palmitate, mandelic acid, sulfur, salicylic acid, tocopherol, plant essential oils, plant functional oils, coenzyme Q10, retinol, ceramide, lutein, totarol, thyroxine, vitamin A, vitamin D, vitamin K, and any combination thereof.

5. The microneedle patch according to claim 4, wherein the fat-soluble functional ingredient and the at least one solid grease of the inner-layer material are mixed uniformly, and a mass ratio of the fat-soluble functional ingredient to the at least one solid grease of the inner-layer material is more than or equal to 0.05:1 and less than 1:1.

6. The microneedle patch according to claim 1, wherein a melting point of the inner-layer material is 25° C. to 40° C.

7. The microneedle patch according to claim 1, wherein the microneedle structure has a microneedle height between 100 μm and 1500 μm, and the base layer has a base-layer height between 5 μm and 300 μm.

8. The microneedle patch according to claim 1, wherein the microneedle outer layer has an outer surface, the microneedle cavity has a sidewall, the microneedle inner layer has a side surface, the side surface of the microneedle inner layer is adjoined to the sidewall of the microneedle cavity, and a wall thickness between the outer surface and the sidewall is between 5 μm and 20 μm.

9. The microneedle patch according to claim 8, wherein a cross-sectional area of the microneedle outer layer parallel to the first surface has a largest outer-layer width from 260 μm to 540 μm, and a cross-sectional area of the microneedle inner layer parallel to the first surface has a largest inner-layer width from 250 μm to 500 μm.

10. The microneedle patch according to claim 1, wherein the microneedle structures are pyramids or cones.
